(19) United States
(12) Patent Application Publication
(10) Pub. No.: US 2005/0191257 A1
(43) Pub. Date: Sep. 1, 2005

(54) Dry deodorant containing a sesquiterpene alcohol and zinc oxide

(76) Inventors: John Brahms, Piscataway, NJ (US); Jairajh Mattai, Piscataway, NJ (US); Ronald Jacoby, Holland, PA (US); Suman Chopra, Dayton, NJ (US); Eric Guenin, Pennington, NJ (US)

Correspondence Address: Colgate-Palmolive Company, 909 River Road, P.O. Box 1343, Piscataway, NJ, 08855-1343, US

(21) Appl. No.: 10/789,153

(22) Filed: Feb. 27, 2004

(51) Int. Cl. A61K 7/32; A61K 7/36

(52) U.S. Cl. 424/67

(57) ABSTRACT

A suspension product for reducing wetness under the arm which product comprises in % by weight based on the entire weight of the product: (a) 0.01-20% of a superabsorbent polymer with water absorbing capacity between 10-1000 g water/g superabsorber in the absence of added sodium chloride; (b) 0.05-10% of a sesquiterpene material; (c) 0.05-10% of a small particle size zinc oxide having a particle size range of 0.02-200 microns; (d) 2-88% of a volatile silicone, wherein the product is not made with any separately added water.

### FIELD OF THE INVENTION

This invention relates to deodorant products containing an effective combination of a superabsorbant polymer, sesquiterpene alcohol and a small particle zinc oxide to provide deodorants with reduced irritation particularly without requiring the use of an antiperspirant active.

### BACKGROUND OF THE INVENTION

Deodorant products typically contain antibacterial actives along with odor masking agents and fragrance to provide odor protection. Current antiperspirant products provide wetness control in addition to odor control due to the presence of antiperspirant salts. Therefore, traditional antiperspirants provide the additional benefit of deodorancy. Deodorants tend to be alkaline with a pH range of 9-10 due to the soap-like base composition. This characteristic can make deodorant irritating to the skin of some individuals. In a similar fashion antiperspirant products can also be irritating, however this is due to the low pH associated with antiperspirant salts. Therefore, an effective neutral pH deodorant is required for sensitive individuals seeking mild odor and wetness control without antiperspirant salts. The present invention delivers superior odor protection without irritation, along with wetness protection.

A variety of technologies have attempted to use superabsorbent polymers of various types in a wide variety of applications. These technologies include the construction of diaper products for children and adults, and the use of superabsorbent polymers to clean up liquid spills. The problems associated with the use of such polymers in personal care applications include a wet and sticky feel and skin irritation. Additionally, it has been difficult to find a way of applying such products in the underarm area in a way that results in an aesthetically acceptable product form. It has now been found that selected water lock superabsorbent polymers in combination with a sesquiterpene alcohol, particularly Farnesol, and an odor absorber, particularly micronized zinc oxide, may be used to provide a product with superior deodorant efficacy along with wetness protection.

Farnesol (3,7,10-trimethyldodeca-2,6,10-trien-1-ol) is an acyclic primary sesquiterpene alcohol, commonly found in lemongrass oil, citronella oil, and sandalwood oil. Farnesol is available from Symrise (Totowa, N.J.) as an ethanol soluble solution. As detailed in U.S. Pat. No. 4,220,665, Farnesol is disclosed as a natural antibacterial agent for use in cosmetic products, which is most effective at inhibiting the growth of Gram positive odor forming bacteria on the skin.

U.S. Pat. No. 5,895,643 describes the combined use of Farnesol in a deodorizing and antimicrobial composition with 2-phenoxyethanol, glycerol monocaprylate, and optionally glycerol monolaurate in deodorizing and anti-microbial compositions.

U.S. Pat. No. 6,444,647 teaches the use of Farnesol as a skin care active in combination with a vitamin B3 compound, and phytantriol in a skin care formulation.

U.S. patent application 2002/0049257 A1 describes antibacterial compositions containing a combination of Farnesol with fractionated components of Sandela in deodorizing personal care products.

U.S. Pat. No. 5,965,518 discloses the use of non-aromatic terpenoids, including Farnesol, in combination with phenolic compounds in an antimicrobial fragrance composition.

U.S. Pat. No. 5,135,747 describes deodorants containing malodor reducing compositions with encapsulated odor fighting agents including cedar wood oil (a sesquiterpene alcohol containing essential oil).

The following article: Nomomura, Mami; Hori, Kimihiko; Ichikawa, Yoshiaki; Fukuda, Kazuyuki; Nojiri, Hiroshi; Takameda, Yoshinori “Research and development of fragrance materials which possess the inhibitory activity towards IL-4 production” Aroma Research, 3, 51-56, 2002, discloses the potential anti-inflammatory effect of certain sesquiterpene alcohols, including Farnesol, guiaol and others.

Japan Patent Number 61033129 discloses pharmaceutical compositions containing sesquiterpene alcohols and polar compounds selected from among the group consisting of cyclic ureas, alkylene glycols, mono and diethylene glycol monoalkyl ethers, lactones and lactams. Such compositions are said to enhance absorption of drug actives through the skin. One example contains n-methylpyrrolidone (74%), bisabalol (23%) and propranolol hydrochloride (1%).

Farnesol is frequently included in non-specific broad coverage of antibacterial agents in numerous patents including; U.S. Pat. No. 6,277,359, U.S. Pat. No. 5,585,092, U.S. Pat. No. 6,086,903 and U.S. patent application 2003/0053970 A1.

U.S. Pat. No. 6,358,499 B2 describes the use of micronized zinc oxide in a low-tack, low-residue dibenzilidene sorbitol (DBS) based deodorant formulations.

U.S. Pat. No. 5,714,447 discloses the application of zinc oxide in combination with a surfactant and a water-soluble zinc-complexing polyamine in a deodorant soap or detergent composition.

U.S. Pat. No. 4,322,308 discloses a deodorant detergent composition comprising a detergent active compound, a deodorant perfume, and a deodorant other than a deodorant perfume, which may be a zinc “salt” such as zinc oxide.

PCT cases WO 99/59538 and 99/59539 from The Boots Company PLC, describe compositions having high surface area zinc oxide having a particle size of 0.1-200 microns, which compositions can be used for absorbing sweat and other bodily liquids or bad breath and body odor, respectively.

U.S. Pat. No. 5,122,418 to Shiseido Company Ltd. teaches a composite powder made with a resin and a coating of, for example zinc oxide, having a particle size of 0.01-1 micron. These powders can be used in deodorants.

U.S. patent application 2002/0146377 A1 to Gillette discloses the use of glass microspheres coated with zinc oxide in deodorant formulations.

In addition, zinc oxide is frequently broadly mentioned in the patent literature as a UV filter and sunscreen agent as in U.S. patent application 2003/0053970 A1, U.S. Pat. No. 6,086,903 and U.S. Pat. No. 6,444,647.

U.S. Pat. Nos. 6,436,382 and 6,426,062 to Colgate-Palmolive, Co. disclose the use of superabsorbant water lock polymers in underarm product formulations which may contain an antiperspirant active and an effective amount of a non-antiperspirant deodorizing agent.

It is also desirable to have the ability to reduce wetness using a deodorant product. While some deodorants contain lesser amounts of antiperspirant actives, there is a segment of the population that prefers to use deodorants that do not contain antiperspirant actives. It would be advantageous to provide a product that reduces wetness particularly without the use of antiperspirant actives.

### BRIEF SUMMARY OF THE INVENTION

The invention comprises an underarm product suitable for use to reduce axillary odor. While the products of the invention may be viewed primarily as deodorants, certain embodiments may also exhibit some antiperspirant effect. In one particular embodiment no additional antiperspirant salt is added. In another embodiment a low level of antiperspirant salt may be included as an optional ingredient. The products of the invention comprise effective combinations of a superabsorbant polymer, a sesquiterpene alcohol (or alternatively an essential oil containing at least 5% or more of a sesquiterpene alcohol, a natural anti-inflammatory antibacterial agent), and a small particle (for example, nanoparticle sized) zinc oxide (which is useful to reduce axillary malodor in addition to underarm wetness). The present invention is particularly advantageous in providing deodorants that have reduced irritation potential in addition to wetness and odor protection without requiring the use of an antiperspirant active.

The underarm products of this invention are suspensions which comprise:
(1) a sesquiterpene alcohol,
(2) a small particle size zinc oxide, particularly a micronized zinc oxide or nanoparticle size zinc oxide having a particle size in the range of 0.02-200 microns,
(3) and a superabsorbent polymer which is a polyacrylate homopolymer (sodium salt) or starch graft copolymer of poly(2-propenamide-co-2-propenioic acid) (sodium salt), with water absorbing capacity between 10-1000 g water/g superabsorber in the absence of added salt such as sodium chloride.

The superabsorbents may be either homopolymers or copolymers (or mixtures thereof) and can be used in a variety of particle sizes, particularly smaller sizes (for example, having 95% of the particles able to go through a 200 mesh screen-comparable to a size of less than 75 microns). The formulations of the present invention are generally deodorants that provide the additional benefit of wetness protection.

### DETAILED DESCRIPTION OF THE INVENTION

Products formulated according to the invention comprise suspension products which may be in the form of sticks, soft solids, roll-ons, pump sprays or aerosol sprays. The products comprise:
(a) 0.01-20 weight % (particularly 0.1-10% and more particularly 0.5-5%) of a superabsorbent polymer selected from the group consisting of a polyacrylate homopolymer (sodium salt) or starch graft copolymers of poly(2-propenamide-co-2-propenioic acid) (sodium salt), with a water absorbing capacity of about 10-1000 g water/g (for example, 50-500 g water/g) superabsorber in the absence of added salt such as sodium chloride;
(b) 0.05-2.0 weight % (particularly 0.1-1.5% and, more particularly, 0.5-1.0%) of a sesquiterpene material selected from the group consisting of: (i) a sesquiterpene alcohol (which itself may be a combination of one or more different sesquiterpene alcohols); (ii) an essential oil containing at least 5% of a sesquiterpene alcohol; or (iii) a combination of (i) and (ii), provided that the level of sesquiterpene alcohol in the final product is at least 0.05% of the total formula;
(c) 0.05-10 weight % (particularly 0.1-5%, more particularly 0.5-4%, and most particularly 1-3% ) of a small particle size zinc oxide, particularly micronized zinc oxide or a nanoparticle size zinc oxide having a particle size range of 0.02-200 microns wherein the small particle size zinc oxide may be incorporated into the present invention in powder form or as a dispersion of small particle size zinc oxide in materials compatible with the present invention, for example esters, oils, and silicones;
(d) 2-88 weight % of a volatile silicone having a flash point of 100 degrees C. or less (especially one or more of hexamethyl disiloxane, D4, D5 and D6 cyclomethicones);
(e) 0-5 weight % of a surfactant with a hydrophilic/lipophilic balance (“HLB value”) in the range of 3-13 (for example, from 0.05-50 weight % (particularly 1-30%) of a silicone copolyol which is 10% in cyclomethicone, or its equivalent may be used for a soft solid);
(f) 0-5 weight % (particularly 1-3%) of an antiperspirant active;
(g) 0-20 weight % (particularly 5-10%) of a nonvolatile silicone having a flash point greater than 100 degrees C.; and
(h) 0-20 weight % (particularly 2-12%) of an emollient (for example, a member selected from the group consisting of C12-15 alkyl benzoate, PEG-8 distearate, PPG-3-myristyl ether, and polyisobutene 250);
(i) 0-5 weight % (particularly 0.5-1.5%) of a fragrance; and
(j) 0-5 weight % (particularly 0.5-2%) of a microencapsulated fragrance.

The fragrance may be encapsulated in a polymer matrix such as a starch, a modified starch, chitosan, cyclodextrin, polyoxymethylene urea, polyoxymethylene melamine, gelatin, or other encapsulating material known to those skilled in the art. The microencapsulate fragrance should be in the form of a power with a mean particle size range of 0.1-500 microns (particularly 1-20 microns); provided that:
(A) if the end product is stick or soft solid, a gelling agent may be added as selected from the group consisting of 5-30 weight % stearyl alcohol; 0.1-10 weight % (on an actives basis) silicone elastomer; 0.1-20 weight % waxes (for example, Japan wax, hydrogenated castor oil); 1-3 weight % siliconized polyamides (especially of the type described below in Formula IIIA); 1-20 weight % low molecular weight polyethylene having a molecular weight in the range of 400-1000 (for example 400 such as Performalene-400 from New Phase Technology, Piscataway, N.J.); and combinations of the foregoing;
(B) if the formulation is an aerosol, a propellant selected from the group consisting of (i) a hydrocarbon (for example, propane, butane, isobutane, and isopentane), (ii) dimethyl ether, (iii) a hydrofluorocarbon (for example, trifluoroethane) or mixtures thereof may be added at a level of 30-90% (See U.S. Pat. No. 3,974,270: Pressurized composition for the production of a dry spray of mild but effective non-staining antiperspirant.);
(C) if the formulation is a roll-on or pump spray, 1-15% of a suspending agent may be added to prevent product separation and may be selected from the group consisting of:
(i) colloidal silica (for example, pyrogenic colloidal silica) with a particle size in the range of 2-100 microns, preferably averaging less than 50 microns in diameter;
(ii) clays selected from the group consisting of montmorillonite clays and hydrophobically treated montmorillonite clays, for example, clays with a particle size in the range of 50-10,000 nanometers;
(iii) magnesium aluminum silicates, for example with a particle size in the range of 0.1-50 microns; and
(iv) mixtures of any of (i)-(iii).

The surfactants useful in this invention have an HLB (hydrophilic-lipophilic balance) value of 3-13. The HLB parameter is a parameter that is well known in the art and explained in numerous references, for example, as described in U.S. Pat. No. 6,682,749.

Zinc oxide in the present invention is used as a low irritation odor absorbing agent with secondary antibacterial activity. Zinc oxide is incorporated as a necessary component of the invention to improve upon the odor control capability of the superabsorbant polymer and the sesquiterpene alcohol.

A sesquiterpene alcohol contains a substructure consisting of 15 carbons. The 15 carbons can be in the form of a linear or branched chain, one or more rings or a combination of rings, fused rings, carbon chains connected completely through carbon-carbon bonds and having at least one hydroxyl group attached. The sesquiterpene alcohol may contain one or more double or triple bonds. The rings can be from 3 to 15 carbons. The rings may be present as the main structure of the molecule or they may be substituents on any carbon within the sesquiterpene system. When more than one ring is present, they may optionally be present as part of a fused or spiro ring system.

In one embodiment, one or more sesquiterpene alcohols (as distinguished from oils containing some portion of a sesquiterpene alcohol as one of several components) are used. Non-limiting examples of sesquiterpene alcohols are as follows:
(i) cyclic sesquiterpene alcohols including but not limited to the following (with CAS numbers in parentheses):
8-α-acetoxyelemol (41370-57-4),
β-acoradienol (149496-35-5),
α-acorenol (28296-85-7),
β-acorenol (28400-11-5),
β-biotol (19902-26-2),
α-bisabolol (25428-43-7),
β-bisabolol (15352-77-9),
bicyclovitivenol (5957-31-3),
β-bisabolenol (147126-90-7),
bulensol (22451-73-6),
α-cadinol (481-34-5),
epi-α-cadinol (11070-72-7),
carotol (465-28-1),
caryophyllene alcohol (4586-22-5),
14-hydroxy-9-epi-e-caryophyllene (79768-25-5),
5-neo-cedranol (13567-44-7),
8-cedren-13-ol (18319-40-9),
cedr-8(15)en-9-α-ol (13567-41-4),
cedrol (77-32-2),
epi-cedrol (19903-73-2),
β-copaen-4-α-ol (126060-41-1),
cryptomeridiol (4666-84-6),
cubebol (23445-02-5),
cubenol (21284-22-0),
1-epi-cubenol (19912-67-5),
1,10-di-epi-cubenol (73365-77-2),
cycloisolongifol-5-ol, dihydrocadinol (1,6-dimethyl-4-iso-propyl-decahydronapthylene) drimenol (468-68-8),
elemol (9639-99-6),
epi-globulol (88-728-589),
α-eudesmol (473-16-5),
β-eudesmol (473-15-4),
γ-eudesmol (1209-71-8),
7-epi-α-eudesmol (123123-38-6),
10-epi-γ-eudesmol (15051-81-7),
dihydro-eudesmol (6770-16-7),
germacrene D-4-ol (74841-87-5),
gleenol (72203-99-7),
guaiol (489-86-1),
globulol (51371-47-2),
gossonorol (92691-77-5),
hexahydrofarnesol (2,6,10-trimethyldodecanol), himachalol (1891-45-8),
hinesol (23811-08),
14-hydroxy-α-humulene (75678-90-9),
14-hydroxy-α-muurolene (135118-51-3),
5-isocedranol (13567-45-8),
isolongifolan-7-α-ol, isolongifolol, khusinol (24268-34-6),
ledol (577-27-5),
z-lancetol (10067-28-4),
longiborneol (465-24-7),
longifolol (1139-17-9),
longipinanol (39703-23-6),
cis-muurol-5-en-4-β-ol, cis-muurol-5-en-4-α-ol, α-muurolol (19435-97-3),
epi-α-muurenol (19912-62-0),
occidentalol (473-17-6),
trans-dihydro-occidentalol, occidol (5986-36-7),
e-nuciferol (39599-18-3),
patchouli alcohol (5986-55-0),
e-β-santalol (37172-32-0),
z-α-santalol (115-71-9),
z-β-santalol (42495-69-2),
β-santalol (37172-32-0),
cis-sesquisabinene hydrate (58319-05-4),
trans-sesquisabinene hydrate (145512-84-1),
z-sesquilavandulol (131485-70-6),
selin-11-en-4-α-ol (16641-47-7),
spirosantalol spathulenol (6750-60-3),
thujopsan-2-α-ol (78392-83-3),
thujopsan-2-β-ol (150737-93-2),
turmenol (38142-57-3),
valerianol (20489-45-6),
viridiflorol (552-2-3),
vitiverol widdrol (6892-80-4),
bicyclo-vetiverol (5957-31-3), and
tricyclovetiverol; and
(ii) a-cyclic sesquiterpene alcohols including but not limited to the following:
dihydrofarnesol, z,z-farnesol (16106-95-9),
e,e-farnesol (106-28-5),
e,z-farnesol (3879-60-5),
e-nerolidol (7212-44-4),
z-nerolidol (142-50-7),
e-sesquilavandulol (120707-27-9),
and tetrahydrofarnesol; and
(iii) one or more sesquiterpene alcohols that have an anti-inflammatory effect as indicated by the ability either in-vitro in a cell culture assay or in-vivo to (A) inhibit prostaglandin cyclooxygenase-I (COX-I), prostaglandin cyclooxygenase-II (COX-II) in-vitro or in-vivo; or (B) the ability to inhibit phospholipase activity, interlukin-1β (II-1β) or interlukin-4 (II-4) (for example, cedrol, α-bisabolol (25428-43-7), β-bisabolol (15352-77-9), guaiol (489-86-1), and e,e-farnesol (106-28-5)).

For an alternate embodiment in which plant essential oils are used wherein such oils are not themselves completely sesquiterpene alcohols but contain a sesquiterpene alcohol component such as at a level of at least 5% of one or more sesquiterpene alcohols, the following list is included:
patchouli wood oil, sandalwood oil, grapefruit oil, lemongrass oil, cedarwood oil, and guiac wood oil;

In another embodiment, mixtures of sesquiterpene alcohols may be used.

In yet another embodiment mixtures of sesquiterpene alcohols and plant essential oils may be used.

One particular group of sesquiterpene alcohols is the group consisting of one or more members of the following group:
epi-globulol, cedrol, patchouli alcohol, z,z-farnesol, e,e-farnesol, e,z-farnesol, guaiol, globulol, e-β-santalol, z-α-santalol, z-β-santalol, α-bisabolol (25428-43-7), and β-bisabolol (15352-77-9).

Colloidal silica is available commercially as, for example, Cab-O-Sil® material, a submicroscopic particulated pyrogenic silica which is a colloidal silica. Among the more preferred pyrogenic silicas the diameters will be in the range of 2 to 20 microns. Often the ultimate particle size of a preferred pyrogenic silica, such as that sold by Cabot Corporation, Boston, Mass., as Cab-O-Sil M-5, will have an ultimate particle diameter of about 11 microns, whereas the corresponding H-5 grade has a diameter of about 7 microns. The surface areas of the pyrogenic colloidal silicas and other colloidal silicas having average particle sizes less than one micron are preferred in this invention, often ranging from about 50 to 500 square meters per gram, leading to their desirable thickening, suspending, and covering properties.

Clay suspension agents suitable for use in the compositions of the present invention are selected from the group consisting of montmorillonite clays and hydrophobically treated montmorillonite clays. Montmorillonite clays are those which contain the mineral montmorillonite and are characterized by having a suspending lattice. Examples of these clays include the bentonites, hectorites, and colloidal magnesium aluminum silicates. Clay materials are typically made hydrophobic by treatment with a cationic surfactant, such as quaternary ammonium cationic surfactants (e.g., ditallow dimethyl ammonium chloride, i.e., quatemium-18).

Bentonite is colloidal, hydrated aluminum silicate obtained from montmorillonite and has the formula Al₂O₃4SiO₂.H₂O. A more detailed discussion of bentonites can be found in the Kirk-Othmer Encyclopedia of Chemical Technology, 2nd. ed., Vol 3(1964), pp 339-360, published by Interscience Publishers, which is incorporated herein by reference.

Hectorite, also a montmorillonite clay, differs from bentonite in that there is almost a complete substitution of aluminum in the lattice structure of bentonite by magnesium. In addition, hectorites contain lithium and fluorine. Laponite™ is an example of a commercially available synthetic hectorite marketed by Laporte Industries, Ltd.

The magnesium aluminum silicates are complexes of colloidal magnesium aluminum silicate richer in magnesium than aluminum. Magnesium aluminum silicates are commercially available as Veegum™ (R. T. Vanderbilt Co.).

Preferred clay suspension agents for use in the present invention include hydrophobically treated montmorillonite clays, e.g., hydrophobic bentonites available under the tradename of Bentone™. Bentone™ is prepared by reacting bentonite in a cation exchange system with an amine. Different amines are reacted to obtain a variety of Bentones, which may also differ in proportions of SiO₂, MgO and Al₂O₄. Specific examples of Bentones™ within the scope of the present invention are Bentone™ 38, Bentone™ 34, Bentone™ 27, Bentone™ 14, and Bentone™ LT, all of which have a particle size of below about 5 microns and are commercially available from the NL Industries, Inc.

The compositions that utilize hydrophobically-treated hectorite and bentonite clays to suspend the antiperspirant active material will also generally include a clay activator. Many such clay activators, as well as the levels of use in liquid antiperspirant compositions, are known in the art. Such activating materials include, for example, propylene carbonate, ethanol, and mixtures thereof. Typically, the level of clay activator will be from about 25% to about 75% of the weight of the clay, more typically from about 40% to about 60% of the weight of the clay. (See EP Application 0 485 012 Al: Liquid antiperspirant composition.)

While no water is recited as being added to the present compositions, up to 2 weight % water may be present because of the types of raw materials used.

With regard to the amount of volatile silicone used in the invention, 10-88 weight % is used for stick products and soft solids.

Optionally, one or more other ingredients can be used such as fragrance, coloring agents, masking agents, or fillers (for example, talc).

Various types of zinc oxide which have small particle sizes can be used in this invention. Small particle zinc oxides that are particularly useful are those in powder form and in dispersions with esters, oils, and silicones compatible with the present invention. Powder forms of zinc oxide may be classified as “micronized” and may have a particle size in the range of 0.02-200 microns, especially in the range of 0.02-10 microns, and most especially 0.1-10 microns. A non-limiting example of a useful micronized zinc oxide powder is Z-COTE® micronized zinc oxide available from BASF (Charlotte, N.C.) which has an average particle size (APS) of 0.2 microns. A silane treated hydrophobic Z-COTE® micronized zinc oxide is also available for oil phase dispersions. Another type of powder form zinc oxide is characterized as being in the “nanoparticle range” with non-limiting examples being NANOX™, nano-sized zinc oxide (untreated) from Elementis Specialties (Hightstown, N.J.). This product has an APS of about 60 nanometers with a broader range of 40-60 or 40-80 nanometers. Also useful is NanoGard® zinc oxide available from NanoPhase Technologies (Romeoville, Ill.) which is a 99.0+% pure zinc oxide powder with an APS of 60 nm. Dispersions of small particle zinc oxide in esters, oils, and silicones are useful in the present invention. Non-limiting examples of useful small particle zinc oxide dispersions include; Nanosun™ Micro zinc oxide “E” 70, which is an ester coated dispersion containing 70% small particle zinc oxide available from Shamrock Technologies Inc. (Newark, N.J.); a high solids dispersion of 20-30 nm zinc oxide (45-50%) in cyclopentasiloxane, PEG-10 dimethicone, and dimethicone available under the trade name of FA50XZ4 from Kobo Products, Inc. (South Plainfield, N.J.); a high solids dispersion of 60 nm zinc oxide (45-50%) in C12-15 alkyl benzoate, triethoxycaprylylsilane, and castor oil phosphate available under the trade name of TNC65FZS from Kobo Products, Inc. (South Plainfield, N.J.); and a high solids dispersion of 100 nm zinc oxide (45-50%) in cyclomethicone, PEG-10 dimethicone, and dimethicone under the trade name of CM3K50LZM also available from Kobo Products, Inc. (South Plainfield, N.J.).

The superabsorbers that can be utilized according to this invention are polyacrylate homopolymers (sodium salt) or starch graft copolymers of poly(2-propenamide-co-2-propenioic acid) (sodium salt), with water absorbing capacity between 10-1000 g water/g superabsorber in the absence of added salt such as sodium chloride. These polyacrylate homopolymers include: Hysorb 8100 (BASF, Charlotte, N.C.), Sanfresh ST-500D, Sanfresh ST-500MPSA, Aquapearl A-3, Aquapearl AP211D (Sanyo Chemical Industries, Japan), AQUA KEEP® J-550, AQUA KEEP® J-500, AQUA KEEP® J-440, AQUA KEEP® 10SH-NF (Kobo Products, Inc., South Plainfield, N.J.). The starch graft copolymers of poly(2-propenamide-co-2-propenioic acid) (sodium salt) are the WATER LOCK® superabsorbers from Grain Processing Corporation, Muscatine, Iowa and include A180 and C200.

One particular embodiment of the invention excludes the use of antiperspirant salts, especially aluminum and aluminum/zirconium salts.

Another embodiment of the invention provides for the use of low levels (such as in the range of less than 5 weight %) of aluminum and/or aluminum/zirconium salts. The antiperspirant salts that can be utilized according to the present invention include conventional aluminum and aluminum/zirconium salts, as well as aluminum/zirconium salts complexed with a neutral amino acid such as glycine (“gly”), as known in the art. See each of European Patent Application Number 512,770 A1 and PCT case WO 92/19221, the contents of each of which are incorporated herein by reference in their entirety, for disclosure of antiperspirant active materials. The antiperspirant active materials disclosed therein, including the acidic antiperspirant materials, can be incorporated in the compositions of the present invention. Suitable materials include (but are not limited to) aluminum chlorohydroxide, aluminum chloride, aluminum sesquichlorohydroxide, zirconyl hydroxychloride, and aluminum chlorohydrol-propylene glycol complex. These include, by way of example (and not of a limiting nature), aluminum chlorohydrate, aluminum chloride, aluminum sesquichlorohydrate, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), and mixtures of any of the foregoing. The aluminum-containing materials can be commonly referred to as antiperspirant active aluminum salts. Generally, the foregoing metal antiperspirant active materials are antiperspirant active metal salts. In the embodiments which are antiperspirant compositions according to the present invention, such compositions need not include aluminum-containing metal salts, and can include other antiperspirant active materials, including other antiperspirant active metal salts. Generally, Category I active antiperspirant ingredients listed in the Food and Drug Administration's Monograph on antiperspirant drugs for over-the-counter human use can be used. In addition, any new drug, not listed in the Monograph, such as tin or titanium analogues of the aluminum slats listed above, aluminum nitratohydrate and its combination with zirconyl hydroxychlorides and nitrates, or aluminum-stannous chlorohydrates, can be incorporated as an antiperspirant active ingredient in antiperspirant compositions according to the present invention. Preferred antiperspirant actives that can be incorporated in the compositions of the present invention include the enhanced efficacy aluminum salts and the enhanced efficacy zirconium/aluminum salt-glycine materials, having enhanced efficacy due to improved molecular distribution, known in the art and discussed, for example, in PCT No. WO 92/19221, the contents of which are incorporated by reference in their entirety herein.

One of the embodiments provides for the optional use of low levels of antiperspirant in the range of 0-5% (on an anhydrous solids basis), preferably 1-3%, by weight, of the total weight of the composition. The amount used will depend on the formulation of the composition. These lower levels will not substantially reduce the flow of perspiration, but will additionally reduce malodor, for example, by acting as a deodorant material, for example, by acting as an antimicrobial or complexing with the malodorous components of human perspiration.

Gelling agents include elastomers such as:
(a) a dimethicone/vinyldimethicone crosspolymer composition made by reacting (in the presence of a platinum catalyst) a polymethylhydrogensiloxane with an alpha, omega-divinylpolydimethyl siloxane for which the dimethicone/vinyldimethicone crosspolymer composition (1) is used at a concentration of 4-10% in cyclomethicone (particularly 4-7%, and, more particularly, 4-6.5%) (for example, where the cyclomethicone is a D5 cyclomethicone), (2) has a refractive index in the range of 1.392-1.402 at 25 degrees C., and (3) has a viscosity in the range of 0.013-1×10⁴ Pascal seconds; for example, one particular elastomer of interest is KSG-15 silicone elastomer from Shin-Etsu Silicones of America (Akron, Ohio).
(b) a cyclomethicone (and) dimethicone crosspolymer made with an ≡Si—H containing polysiloxane and an alpha, omega-diene of formula CH₂═CH(CH₂)ₓCH═CH₂, where x=1-20, to form a gel by crosslinking and addition of ≡Si—H across double bonds in the alpha, omega diene, which crosspolymer has a viscosity in the range of 50,000-3,000,000 centipoise (particularly 100,000-1,000,000; more particularly 250,000-450,000 centipoise; and most particularly 350,000 centipoise), preferably with a nonvolatiles content of 8-18% (particularly 10-14% and most particularly 12-13%) in cyclomethicone (for example a D5 cyclomethicone), (an example of such a crosspolymer composition being DC-9040 from Dow Coming Corporation (Midland, Mich.) with other types of such crosspolymers (also called elastomers) being described in U.S. Pat. No. 5,654,362, incorporated by reference herein as to the description of such polymers and methods of making such polymers);

Particular examples of suitable elastomers are SFE 167, a cetearyl dimethicone/vinyl dimethicone crosspolymer from GE Silicones (Waterford, N.Y.); SFE 168, a cyclomethicone (and) dimethicone/vinyl dimethicone crosspolymer from GE Silicones; vinyl dimethicone crosspolymers such as those available from Shin-Etsu Silicones of America (Akron, Ohio) under trade names KSG-15 (cyclomethicone (and) dimethicone/vinyl dimethicone crosspolymer), KSG-16 (dimethicone (and) dimethicone/vinyl dimethicone crosspolymer), KSG-17 (cyclomethicone (and) dimethicone/vinyl dimethicone crosspolymer), KSG-18 (phenyl trimethicone (and) dimethicone/phenyl vinyl dimethicone crosspolymer); and KSG-20 (dimethicone copolyol crosspolymer; dimethicone/vinyl dimethicone crosspolymer from Dow Coming Corporation (Midland, Mich.) under trade name Dow Coming 9506 Cosmetic Powder, DC-9040 elastomer in cyclomethicone from Dow Coming; and a mixture of cyclomethicone and stearyl-vinyl/hydromethylsiloxane copolymer available from Grant Industries, Inc. (Elmwood Park, N.J.) under the trade name Gransil SR-CYC.

The gelling agent may include both high and low melting point waxes. An example of such a combination of waxes includes 5-23 percent stearyl alcohol and 2-8 percent Japan wax. A second example is 5-23% stearyl alcohol, 2-8% hydrogenated castor oil and 2-8% PEG-8 distearate. A third example is 5-15% polyethylene, 2-8% Japan wax and 10-40% silicone elastomer (KSG-15).

For gelling agents which are polyamides, one should include at least one siliconized polyamide of Formula IIIA: 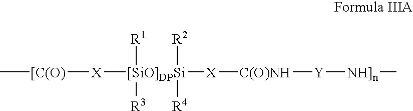
where:
(1) DP is a number in the range of 10-40 (particularly 15-30);
(2) n is a number selected from the group consisting of 1-500;
(3) X is a linear or branched chain alkylene having 1-30 carbons;
(4) Y is selected from the group consisting of linear and branched chain alkylenes having 1-40 carbons, wherein:
(A) the alkylene group may optionally and additionally contain in the alkylene portion at least one of the members of a group consisting of (i) 1-3 amide linkages; (ii) C5 or C6 cycloalkane (as a cycloalkylene linkage); and (iii) phenylene optionally substituted by 1-3 members selected independently from the group consisting of C1-C3 alkyls; and
(B) the alkylene group itself may optionally be substituted by at least one member selected from the group consisting of (i) hydroxy; (ii) C3-C8 cycloalkyl; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine; or Y═Z² where 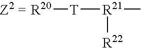
   wherein each of R²⁰, R²¹ and R²² are independently selected from the group consisting of linear and branched C1-C10 alkylenes; and T is selected from the group consisting of (i) a trivalent atom selected from N, P and Al; and (ii) —CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl, especially methyl and ethyl and most especially methyl; and
(5) each of R¹—R⁴ is independently selected from the group consisting of methyl, ethyl, propyl, isopropyl, a siloxane chain, and phenyl, wherein the phenyl may optionally be substituted by 1-3 members from the group consisting of methyl and ethyl (with more particular values for R¹—R⁴ being selected from methyl and ethyl and especially methyl);
wherein the polyamide of Formula IIIA has:
   (i) a silicone portion in the acid side of the polyamide;
   (ii) a degree of polymerization in the range of 10-40 (particularly 15-30);
   (iii) an average molecular weight of at least 50,000 daltons (particularly in the range of 80,000-150,000 daltons and, more particularly in the range of 90,000-120,000 daltons) with at least 95% of the polyamide having a molecular weight greater than 10,000 daltons; and
   (iv) a polydispersity of less than 20 (particularly less than 4).

One particular set of embodiments includes a gelling agent selected from the group consisting of 5-30 weight % stearyl alcohol; 0.1-10 weight % (on an actives basis) silicone elastomer; 0.1-20 weight % waxes; and 0.1-20 weight % low molecular weight polyethylene having a molecular weight in the range of 400-1000.

Volatile silicones and silicone surfactants are also used in the invention.

By volatile silicone material is meant a material that has a flash point of 100 degrees C. or less at atmospheric pressure. Such volatile silicones include conventional cyclic and linear volatile silicones such as cyclomethicone (especially cyclopentasiloxane, also called “D5”), “hexamethyldisiloxane”, and low viscosity dimethicone (for example, Dow Corning® 200 fluid having a viscosity of 0.5-5 centistokes). Illustratively, and not by way of limitation, the volatile silicones are one or more members selected from the group consisting of cyclic polydimethylsiloxanes such as those represented by Formula III-S: 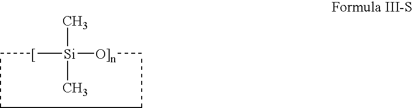
where n is an integer with a value of 3-7, particularly 5-6. For example, DC-245 fluid (or the DC-345 version) from Dow Coming Corporation (Midland, Mich.) is a type of cyclomethicone which can be used. These include a tetramer (or octylmethylcyclotetrasiloxane) and a pentamer (or decamethylcyclopentasiloxane). The volatile linear silicones can also be included in this group of volatile silicones and are one or more members selected from the group consisting of linear polydimethylsiloxanes such as those represented by Formula IV-S: 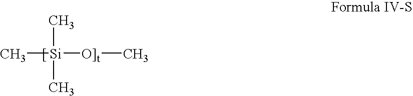
and t is selected to obtain a viscosity of 0.5-5 centistokes.

Examples of such volatile silicones include one or more members selected from the group consisting of D4, D5, and D6 cyclomethicones; and linear dimethicones having a viscosity in the range of 0.5-10 centistokes. Preferably the oil phase is a mixture of one or more of D4, D5 and D6 cyclomethicones.

Suitable silicone surfactants include silicone polyglucosides (for example, octyl dimethicone ethoxy glucoside) and silicone copolyols having an HLB value (hydrophilic lipophilic balance) in the range of 3-13. A silicone copolyol (especially dimethicone copolyol) may be used in an amount of 0.05-5.0 weight % (actives basis), particularly 0.1-3.0% and, more particularly, 0.1-2.0%.

In general, silicone copolyols useful in the present invention include copolyols of the following Formulae I-S and II-S. Formula I materials may be represented by:
(R¹⁰)₃—SiO—[(R¹¹)₂—SiO]ₓ—[Si(R¹²)(R^{b}—O—(C₂H₄O)ₚ—(C₃H₆O)ₛ—R^{c})O]_{y}—Si—(R¹³)₃ Formula I-S
wherein each of R¹⁰, R¹¹, R¹² and R¹³ may be the same or different and each is selected from the group consisting of C1-C6 alkyl; R^{b} is the radical —CₘH₂ₘ—; R^{c} is a terminating radical which can be hydrogen, an alkyl group of one to six carbon atoms, an ester group such as acyl, or an aryl group such as phenyl; m has a value of two to eight; p and s have values such that the oxyalkylene segment —(C₂H₄O)ₚ—(C₃H₆O)ₛ— has a molecular weight in the range of 200 to 5,000; the segment preferably having fifty to one hundred mole percent of oxyethylene units —(C₂H₄O)ₚ— and one to fifty mole percent of oxypropylene units —(C₃H₆O)ₛ—; x has a value of 8 to 400; and y has a value of 2 to 40. Preferably each of R¹⁰, R¹¹, R¹² and R¹³ is a methyl group; R^{c} is H; m is preferably three or four whereby the group R^{b} is most preferably the radical —(CH₂)₃—; and the values of p and s are such as to provide a molecular weight of the oxyalkylene segment —(C₂H₄O)ₚ—(C₃H₆O)ₛ— of between about 1,000 to 3,000. Most preferably p and s should each have a value of about 18 to 28.

A second siloxane polyether (copolyol) has the Formula II-S:
(R¹⁰)₃—SiO—[(R¹¹)₂—SiO]ₓ—[Si(R¹²)(R^{b}—O—(C₂H₄O)ₚ—R^{c})O]_{y}—Si—(R¹³)₃ Formula II-S
wherein p has a value of 6 to 16; x has a value of 6 to 100; and y has a value of 1 to 20 and the other moieties have the same definition as defined in Formula I-S.

It should be understood that in both Formulas I and II shown above that the siloxane-oxyalkylene copolymers of the present invention may, in alternate embodiments, take the form of endblocked polyethers in which the linking group R^{b}, the oxyalkylene segments, and the terminating radical R^{c} occupy positions bonded to the ends of the siloxane chain, rather than being bonded to a silicon atom in the siloxane chain. Thus, one or more of the R¹⁰, R¹¹, R¹² and R¹³ substituents which are attached to the two terminal silicon atoms at the end of the siloxane chain can be substituted with the segment —R^{b}—O—(C₂H₄O)ₚ—(C₃H₆O)ₛ—R^{c} or with the segment —R^{b}—O—(C₂H₄O)ₚ—R^{c}. In some instances, it may be desirable to provide the segment —R^{b}—O—(C₂H₄O)ₚ—(C₃H₆O)ₛ—R^{c} or the segment —R^{b}—O—(C₂H₄O)ₚ—R^{c} at locations which are in the siloxane chain as well as at locations at one or both of the siloxane chain ends.

Particular examples of suitable dimethicone copolyols are available either commercially or experimentally from a variety of suppliers including Dow Corning Corporation, Midland, Mich.; General Electric Company, Waterford, N.Y.; Witco Corp., Greenwich, Conn.; and Goldschmidt Chemical Corporation, Hopewell, Va. Examples of specific products include DOW CORNING® 5225C from Dow Corning which is a 10% dimethicone copolyol in cyclomethicone; DOW CORNING® 2-5185C which is a 45-49% dimethicone copolyol in cyclomethicone; SILWET L-7622 from Witco; ABIL EM97 from Goldschmidt which is a 85% dimethicone copolyol in D5 cyclomethicone; and various dimethicone copolyols available either commercially or in the literature.

It should also be noted that various concentrations of the dimethicone copolyols in cyclomethicone can be used. While a concentration of 10% in cyclomethicone is frequently seen commercially, other concentrations can be made by stripping off the cyclomethicone or adding additional cyclomethicone. The higher concentration materials such as DOW CORNING® 2-5185 material is of particular interest.

In one particular embodiment 0.5-50 weight % (particularly 10-30%) of a 10% silicone copolyol such as dimethicone copolyol in cyclomethicone mixture may be used, wherein the amount of mixture added is selected so that the level of silicone copolyol in the cosmetic composition is in the range of 0.05-5.0% (particularly 0.1-3.0%).

Non-volatile silicones may also be used in the formulations of this invention. Such nonvolatile silicones have a flash point greater than 100 degrees C. and a viscosity in the range of 6-1000 centistokes. Suitable non volatile silicones include linear organo-substituted polysiloxanes which are polymers of silicon/oxygen with a general structure:
(1) (R¹⁰)₃SiO(Si(R¹¹)₂O)ₓSi(R¹²)₃ where R¹⁰, R¹¹ and R¹² can be the same or different and are each independently selected from the group consisting of phenyl and C1-C60 alkyl; or
(2) HO(R¹⁴)₂SiO(Si(R¹⁵)₂O)ₓSi(R¹⁶)₂OH, where R¹⁴, R¹⁵ and R¹⁶ can be the same or different and are each independently selected from the group consisting of phenyl and C1-C60 alkyl.

Specific examples include dimethicone, dimethiconol behenate, C₃₀₋₄₅ alkyl methicone, stearoxytrimethylsilane, phenyl trimethicone and stearyl dimethicone.

Emollients are a known class of materials in this art, imparting a soothing effect to the skin. These are ingredients that help to maintain the soft, smooth, and pliable appearance of the skin. Emollients are also known to reduce whitening on the skin and/or improve aesthetics. Examples of chemical classes from which suitable emollients can be found include:
(a) fats and oils which are the glyceryl esters of fatty acids, or triglycerides, normally found in animal and plant tissues, including those which have been hydrogenated to reduce or eliminate unsaturation. Also included are synthetically prepared esters of glycerin and fatty acids. Isolated and purified fatty acids can be esterified with glycerin to yield mono-, di-, and triglycerides. These are relatively pure fats which differ only slightly from the fats and oils found in nature. The general structure may be represented by Formula III: 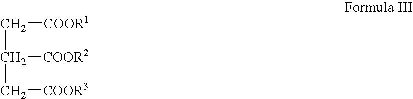
   wherein each of R¹, R², and R³ may be the same or different and have a carbon chain length (saturated or unsaturated) of 7 to 25. Specific examples include peanut oil, sesame oil, avocado oil, coconut, cocoa butter, almond oil, safflower oil, corn oil, cotton seed oil, castor oil, hydrogenated castor oil, olive oil, jojoba oil, cod liver oil, palm oil, soybean oil, wheat germ oil, linseed oil, and sunflower seed oil;
(b) hydrocarbons which are a group of compounds containing only carbon and hydrogen. These are derived from petrochemicals. Their structures can vary widely and include aliphatic, alicyclic and aromatic compounds which have 7-40 carbons. Specific examples include paraffin, petrolatum, hydrogenated polyisobutene, and mineral oil;
(c) esters which chemically are the covalent compounds formed between acids and alcohols. Esters can be formed from almost all acids (carboxylic and inorganic) and any alcohol. Esters here are derived from carboxylic acids and an alcohol. The general structure would be R⁴CO—OR⁵. The total number of carbons for R⁴ and R⁵ together can vary from 7 to 40 and can be saturated or unsaturated, straight chained or branched or can include an aromatic structure. Specific examples include isopropyl myristate, isopropyl palmitate, isopropyl stearate, isopropyl isostearate, butyl stearate, octyl stearate, hexyl laurate, cetyl stearate, diisopropyl adipate, isodecyl oleate, diisopropyl sebacate, isostearyl lactate, C₁₂₋₁₅ alkyl benzoates, myreth-3 myristate, dioctyl malate, neopentyl glycol diheptanoate, neopentyl glycol dioctanoate, dipropylene glycol dibenzoate, C₁₂₋₁₅ alcohols lactate, isohexyl decanoate, isohexyl caprate, diethylene glycol dioctanoate, octyl isononanoate, isodecyl octanoate, diethylene glycol diisononanoate, isononyl isononanoate, isostearyl isostearate, behenyl behenate, C₁₂₋₁₅ alkyl fumarate, laureth-2 benzoate, propylene glycol isoceteth-3 acetate, propylene glycol ceteth-3 acetate, octyldodecyl myristate, cetyl ricinoleate, myristyl myristate (with a particular ester of interest being C12-15 alkyl benzoate);
(d) saturated and unsaturated fatty acids which are the carboxylic acids obtained by hydrolysis of animal or vegetable fats and oils. These have general structure R⁶COOH with the R⁶ group having a carbon chain length of 7-25 and R⁶ can be straight chain or branched. Specific examples include lauric, myristic, palmitic, stearic, oleic, linoleic and behenic acid;
(e) saturated and unsaturated fatty alcohols (including guerbet alcohols) with general structure R⁷COH where R⁷ can be straight chain or branched and have a carbon chain length of 7 to 30. Specific examples include lauryl, myristyl, cetyl, isocetyl, stearyl, isostearyl, oleyl, ricinoleyl and erucyl alcohol;
(f) lanolin and its derivatives which are a complex esterified mixture of high molecular weight esters of (hydroxylated) fatty acids with aliphatic and alicyclic alcohols and sterols. General structures would include R⁸CH₂—(OCH₂CH₂)ₙOH where R⁸ represents the fatty groups derived from lanolin and n=5 to 75 or R⁹CO—(OCH₂CH₂)ₙOH where R⁹CO— represents the fatty acids derived from lanolin and n=5 to 100. Specific examples include lanolin, lanolin oil, lanolin wax, lanolin alcohols, lanolin fatty acids, isopropyl lanolate, ethoxylated lanolin and acetylated lanolin alcohols;
(g) alkoxylated alcohols wherein the alcohol portion is selected from aliphatic alcohols having 2-18 and more particularly 4-18 carbons, and the alkylene portion is selected from the group consisting of ethylene oxide, and propylene oxide having a number of alkylene oxide units from 2-53 and, more particularly, from 2-15. Examples include cetyl glyceryl ether, isostearyl glyceryl ether, isostearyl glyceryl pentaerythrityl ether, laureth-5 butyl ether, oleyl glyceryl ether, PEG-4 ditallow ether, polyglyceryl-3 cetyl ether, polyglyceryl-4 lauryl ether, PPG-9 diglyceryl ether, and propylene glycol myristyl ether. More specific examples include PPG-14 butyl ether, PPG-53 butyl ether, laureth-5 butyl ether, and PEG-4 ditallow ether;
(h) ethers selected from the group consisting of dicapryl ether, dicetyl ether, dimethyl ether, distearyl ether, ethyl ether, isopropyl hydroxycetyl ether, methyl hexyl ether, and polyvinyl methyl ether;
(i) adipic acid blends selected from the group consisting of trimethyl pentanediol/adipic acid copolymer (LEXOREZ TL8 from Inolex, Philadelphia, Pa.), trimethyl pentanediol/adipic acid/isononanoic acid copolymer (LEXOREZ TC8), and adipic acid/diethylene glycol/glycerin crosspolymer (LEXOREZ 100); and
(j) mixtures and blends of two or more of the foregoing.

One particular group of emollients includes C12-15 alkyl benzoate (FINSOLV TN from Finetex Inc., Elmwood Park, N.J.), medium volatility dimethicone (especially 10-350 centistoke material and more especially 10-200 centistoke material), isopropyl myristate; and neopentyl glycol diheptanoate.

Particular examples of suitable emollients include members of the group consisting of Octyloxyglycerin (SENSIVA SC50 from Schülke Mayr, Nordstedt, Germany) (which can be used as an emollient as well as an antibacterial); ethoxylated alcohols such as steareth-2, nonoxynol-2, PPG-4-Ceteth-1; ethoxylated carboxylic acids such as PEG-4 dilaurate, PEG-2 oleate; glyceryl esters such as PEG-2 castor oil, polyglyceryl-3 oleate, glyceryl stearate; sorbitan derivatives such as sorbitan oleate; PPG-3 myristyl ether (such as WITCONOL APM from Goldschmidt); a dimethiconol (such as Dow Corning® DC 1501 dimethiconol); neopentyl glycol diheptanoate; PEG-8 laurate, isocetyl stearate; isostearyl isostearate; isostearyl palmitate; isostearyl alcohol; PPG-5-ceteth-20; PPG-10-cetyl ether; triethyl hexanoin; ethyl hexyl isostearate, glyceryl oleate, and isopropyl isostearate.

The emollient or emollient mixture or blend thereof incorporated in compositions according to the present invention can, illustratively, be included in amounts of 1-15%, and particularly 3-12% by weight of the total weight of the composition.

The compositions of this invention include sticks, soft solids, roll-ons, pump sprays or aerosol sprays. The compositions of the invention may range in clarity from opaque to white.

While one embodiment is fragrance-free, other embodiments contemplate the use of fragrances. In those cases, fragrances may be included such as in an amount of 0-5 weight %, more preferably 0.05-2.5% more preferably 0.5-1.5%, especially a mixture of volatile organic compounds which impart a pleasant odor to the product and mask base odor of the product formulation, if any. It is assumed that the fragrance may impart benefits to the consumer such as masking of underarm or other body odors or bacteriostatic effect. Fragrances consist of complex mixtures of volatile organic compounds and natural essential oils including but not limited to ones known to those skilled in the art. Examples of materials commonly used in formulations disclosed in this invention include but are not limited to those described in “Perfume and Flavor Chemicals” by Steffan Arctander, published by Allured Publishing Company, Carol Stream, III, 2000.

The fragrance may be added directly into the product during the manufacturing process as a free oil or may be either partly or completely microencapsulated prior to adding to the product formulation. The encapsulated portion of the fragrance, if any, may be the same or different in composition to the fragrance free oil and may be present at a level of 0-5 weight % (particularly 0.5-2%). There are many benefits of encapsulating fragrance and the practice is well known to those skilled in the art including but not limited to protecting volatile or reactive fragrance materials from loss during manufacturing or storage, controlled release of fragrance during or after product application through exposure of the encapsulated fragrance to moisture, pH change or mechanical shear. There are numerous methods by which fragrances can be encapsulated well known to those skilled to the art. Non-limiting examples of encapsulation materials and methods suitable for encapsulation of fragrances to be used in compositions of this invention are disclosed in Jens Uhlmann, Brigit Schleifenbaum, Heinz-Jurgen Bertram, “Flavor encapsulation technologies: an overview including recent developments.” Perfumer and Flavorist, 27, 52-61, 2002 and “Selection of Coating and Microencapsulation Processes” by Robert E. Sparks and Irwin Jacobs in Controlled-Release Delivery Systems for Pesticides, Herbert B. Scher ed., Marcel Dekker, New York, N.Y., 1999, pp 3-29.

Various embodiments of the invention may be contemplated. Examples of these include the following.

For stick products, the following general amounts of ingredients may be used:

Formulation A

(a) 8-25 weight % (particularly 8-20%) superabsorbent polymer as described above;
(b) 0.05-10 weight % (particularly 0.5-2%) of a sesquiterpene material (such as sesquiterpene alcohol or mixture of sesquiterpene alcohols or an essential oil containing greater than 5% sesquiterpene alcohol);
(c) 0.05-10 weight % (particularly 0.5-4%) of a small particle size zinc oxide which may be a powder form of zinc oxide classified as “micronized” and having a particle size in the range of 0.02-200 microns, especially in the range of 0.02-10 microns, and most especially 0.1-10 microns. Alternatively nanoparticle powder zinc oxide may be used and is characterized as being in the “nanoparticle range” ranging from 20-1000 nanometers. Dispersions of micronized and nanoparticulate zinc oxide in esters, oils, and silicones are also used in the present invention.
(d) 10-25 weight % of a gellant (for example, selected from the group consisting of silicone elastomer of the type described above (for example, KSG-15 from Shin-Etsu or DC 9040 from Dow Coming), stearyl alcohol, waxes (both low and/or high melting point waxes), hydrogenated castor oil, and low molecular weight polyethylene (such as a molecular weight of about 400 for example, Performalene-400);
(e) 40-70 weight % of a volatile silicone selected from the group consisting of a cyclomethicone (for example, one or more of D4, D5 or D6);
(f) 0-15 weight % of a non-volatile silicone which is a dimethicone having a viscosity in the range of 6-1000 centistokes;
(g) 2-10 weight % of an emollient selected from the group consisting of polyisobutene, and C 12-15 alkyl benzoates (such as FINSOLV TN);
(h) 0-5 weight %, (particularly 0.5-1.5%) fragrance;
(i) 0-5 weight % (particularly 0.5-2%) of a microencapsulated fragrance;
(j) 0-10 weight % (particularly 1-5%) surfactants (for example, PEG-8 distearate or PPG-3 myristyl ether); and
(k) less than 2 weight % water.

For soft solid products, the following general amounts of ingredients may be used:

Formulation B

(a) 70-99.94 weight % silicone elastomer of the type described above (for example, KSG-15 or DC 9040);
(b) 0.01-30 weight % superabsorbent of the type described above;
(c) 0.05-10 weight % (particularly 0.5-2%) of a sesquiterpene material (such as sesquiterpene alcohol or mixture of sesquiterpene alcohols or an essential oil containing greater than 5% sesquiterpene alcohol);
(d) 0.05-10 weight % (particularly 0.5-4%) of a small particle size zinc oxide which may be a powder form of zinc oxide classified as “micronized” and having a particle size in the range of 0.02-200 microns, especially in the range of 0.02-10 microns, and most especially 0.1-10 microns. Alternatively nanoparticle powder zinc oxide may be used and is characterized as being in the “nanoparticle range” ranging from 20-1000 nanometers. Dispersions of micronized and nanoparticulate zinc oxide in esters, oils, and silicones are also used in the present invention;
(e) 0-5 weight % (particularly 0.5-1.5%) fragrance;
(f) 0-5 weight % (particularly 0.5-2%) of a microencapsulated fragrance;
(g) less than 2 weight % water.

For roll-on or pump spray products, the following general amounts of ingredients may be used:

Formulation C

(a) 0-80 weight % of a volatile silicone;
(b) 0-50 weight % silicone elastomer of the type described above (for example, KSG-15 or DC 9040);
(c) 0-80 weight % of a non-volatile silicone which is a dimethicone having a viscosity in the range of 6-1000 centistokes;
(d) 0.01-30 weight % superabsorbent of the type described above;
(e) 0.05-10 weight % (particularly 0.5-2%) of a sesquiterpene material (such as sesquiterpene alcohol or mixture of sesquiterpene alcohols or an essential oil containing greater than 5% sesquiterpene alcohol);
(f) 0.05-10 weight % (particularly 0.5-4%) of a small particle size zinc oxide which may be a powder form of zinc oxide classified as “micronized” and having a particle size in the range of 0.02-200 microns, especially in the range of 0.02-10 microns, and most especially 0.1-10 microns. Alternatively nanoparticle powder zinc oxide may be used and is characterized as being in the “nanoparticle range” ranging from 20-1000 nanometers. Dispersions of micronized and nanoparticulate zinc oxide in esters, oils, and silicones are also used in the present invention;
(g) 0-5 weight % (particularly 0.5-1.5%) fragrance;
(h) 0-5 weight % (particularly 0.5-2%) of a microencapsulated fragrance;
(i) 1-10% of a suspending agent; and
(j) less than 2 weight % water.

For aerosol spray products, the following general amounts of ingredients may be used:

Formulation D

(a) 30-80 weight % of a volatile silicone;
(b) 30-90% of an aerosol propellant;
(c) 0.01-30 weight % superabsorbent of the type described above;
(d) 0.05-10 weight % (particularly 0.5-2%) of a sesquiterpene material (such as sesquiterpene alcohol or mixture of sesquiterpene alcohols or an essential oil containing greater than 5% sesquiterpene alcohol);
(e) 0.05-10 weight % (particularly 0.5-4%) of a small particle size zinc oxide which may be a powder form of zinc oxide classified as “micronized” and having a particle size in the range of 0.02-200 microns, especially in the range of 0.02-10 microns, and most especially 0.1-10 microns (alternatively nanoparticle powder zinc oxide may be used and is characterized as being in the “nanoparticle range” ranging from 20-1000 nanometers; dispersions of micronized and nanoparticulate zinc oxide in esters, oils, and silicones may also used in the present invention);
(f) 0-5 weight %, particularly 0.5-1.5% fragrance;
(g) 0-5 weight % (particularly 0.5-2%) of a microencapsulated fragrance;
(h) 1-0% of a suspending agent; and
(i) less than 2 weight % water.

### EXAMPLES

The following Examples are offered as illustrative of the invention and are not to be construed as limitations thereon. In the Examples and elsewhere in the description of the invention, chemical symbols and terminology have their usual and customary meanings. In the Examples as elsewhere in this application values for n, m, etc. in formulas, molecular weights and degree of ethoxylation or propoxylation are averages. Temperatures are in degrees C. unless otherwise indicated. The amounts of the components are in weight percents based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997).

### Example A

### Underarm Deodorancy Panel Test

Bacterial viability and panelist's underarm odor assessments were conducted with the “Dry Deodorant” stick containing 10% superabsorbant polymer, 1.0% farnesol and 2.0% zinc oxide, a “Dry Deodorant” stick containing 10% superabsorbant polymer without farnesol or zinc oxide, and a currently commercialized sodium stearate based deodorant stick. All three test products were unfragranced. A cup scrub collection method (P. Williamson and A. M. Kligman, J. Investig. Dermatol. 45: 498-503, (1965) was used to perform underarm bacterial sampling. Briefly, male panelists (n=24) were selected and instructed to wash with a non-antibacterial bar soap for 1 week. During this washout period, panelists were instructed to refrain from the use of underarm products. Following the 1-week washout period, panelists' axillae were sampled via the cup-scrub method to generate a baseline bacterial count. In this method, the area of the axilla to be sampled was delineated using a sterile glass-sampling cylinder. The cylinder was pressed firmly against the skin surface of the axilla during sampling to ensure that the washing fluid did not leak from the sampling site. A 1.5 ml sample of washing fluid (0.1 M phosphate buffer with 0.1% Triton X-100, pH=7.9) was pipetted into the cylinder. The area of skin being sampled was scrubbed with moderate pressure for one minute using a sterile glass rod. After scrubbing the fluid was removed using a sterile pipette and placed into a tared glass vial. The cup was maintained in the same position and the sample collection was repeated on the identical site with an additional 1.5 ml of wash fluid. The solutions were pooled and then standardized to a sample weight of 4.0 grams with additional collection buffer solution. The panelists were then randomly provided with 2 of 3 test products to use for a period of one week. During the one-week product use period, panelists were also asked to assess their axillae for underarm odor and score the odor on a scale of 1-7, 7 being the most malodorous. At the completion of the one-week product use period the panelist's axillae were again sampled using the cup-scrub procedure to ascertain bacterial growth.

Cultures for the total bacterial counts were prepared by the agar incorporation method (pour-plate method). The plates are cooled overnight and then incubated at 37 degrees C. for 48 hours. The number of colony forming units (CFU's) was determined using an automated plate reader.

Results:

The results showed that the “Dry deodorant” without any antimicrobial or odor control active enhanced the growth of bacteria following usage (Table 2). In contrast, both the commercialized deodorant stick and the “Dry Deodorant” with 10% superabsorbant polymer, 1.0% farnesol and 2.0% zinc oxide showed significant reduction in bacteria over the “Dry Deodorant” containing only 10% superabsorber. The bacterial log reduction for the commercial deodorant base and the “Dry Deodorant” containing 10% superabsorbant polymer, 1.0% farnesol and 2.0% zinc oxide were at statistical parity. Similar results were obtained for panelist self-assessment for odor (Table 2). Thus, the combination of the superabsorbant polymer, farnesol and zinc oxide is able to provide excellent odor control in a neutral pH “Dry Deodorant” stick formulation.

TABLE 1
Compositions of a typical Dry Deodorant Stick with
and without ZnO/farnesol

| | Deodor. Stick A | Deodor. Stick B |
|---|---|---|
| Ingredient | % weight | % weight |
| Cyclomethicone (DC 345) | 39.00 | 36.00 |
| Stearyl alcohol | 20.00 | 20.00 |
| C12-C15 alkyl benzoate | 12.00 | 12.00 |
| Q2-5244 (dimethicone copolyol) | 1.00 | 1.00 |
| Hydrogenated castor oil; MP 80 | 4.00 | 4.00 |
| Water Lock ® | 10.00 | 10.00 |
| Superabsorbent, A180 | | |
| PEG-8 distearate | 4.00 | 4.00 |
| DC9040 | 8.00 | 8.00 |
| Japan Wax 525 | 2.00 | 2.00 |
| Farnesol (Dragoco) | | 1.00 |
| Micronized ZnO (Rheox) | | 2.00 |
| Total | 100.00 | 100.00 |

TABLE 2
Log Reduction of bacteria, from baseline, and Odor Score
after 1 week product usage for commercial deodorant Base,
Dry deodorant and Dry deodorant + 1.0%
farnesol + 2.0% zinc oxide

| | |
|---|---|
| Log Reduction | Odor Score |

Product

| | | |
|---|---|---|
| Commercial Deodorant (C) | 1.25 (A) | 2.4 (A) |
| Dry deodorant (A) | −0.14 (B) | 3.7 (B) |
| Dry deodorant + 1% farnesol + 2.0% | 0.71 (A) | 1.9 (A) |
| zinc oxide (B) | | |
| Statistical | | |
| Comparison | | |
| C vs. A | P < 0.001 | P < 0.02 |
| B vs. C | P = 0.08 | P > 0.37 |
| B vs. A | P < 0.05 | P < 0.001 |

C is a commercial product, Speed Stick Ultimate Deodorant from Colgate Palmolive Company.

### Examples 1, 3, and 4

### Stick Product

A stick product of about 400 grams can be made using the ingredients listed in Table 3. The dimethicone (DC 200, 10 censtistokes from Dow Corning Corporation, Midland, Mich.) and C12-15 alkyl benzoate (FINSOLV TN, from Finetex Elmwood Park, N.J.) (and polyisobutene and PPG-3 myristyl ether for Example 3) are added to a suitable size first beaker and heated with stirring to 55-60 degrees C. The Japan wax substitute 525 is added and mixed until melted. The temperature is increased to 82-85 degrees C. and the low molecular weight polyethylene (Performalene-400 from New Phase Technologies, Piscataway, N.J.) is added and mixed until melted. The mixture is then cooled to a temperature of about 80 degrees C. In a separate second beaker the silicone elastomer (KSG-15 from Shin-Etsu Silicones of America, Akron, Ohio) is added followed by the addition of the cyclomethicone (Cyclomethicone 245 from Dow Corning Corporation, Midland, Mich.). The mixture is stirred for about 5 minutes and then heated to a temperature of about 70 degrees C. The silicone elastomer/cyclomethicone mixture from the second beaker is then added to the first beaker with continuous stirring while maintaining the temperature at 78-80 degrees C. The superabsorbent material (Water Lock® Superabsorbent Polymer, A 180 from Grain Processing Corporation, Muscatine, Iowa or Hysorb 8100 (BASF, Charlotte, N.C.), ground to a particle size less than 75 micron, and the antiperspirant active (if used) (active as described in Example 3) are then added at this temperature and stirred for 10 minutes. Zinc oxide is added with stirring for about 5 minutes. Farnesol, fragrance and encapsulated fragrance (if used) as listed in Table 3 are finally added at 78-80 degrees C. temperature and stirred for 1 minute. The product is poured into suitable containers (size is approximately 3 cm (width at widest part of oval)×6 cm (length of base)×10 cm (height) with an ovoid shape) at 78-80 degrees C. and cooled for 15 minutes in a refrigerator at about 4 degrees C. and then at room temperature.

### Example 2

### Stick Product

A stick product of about 400 grams may be made using the ingredients listed in Table 3. The cyclomethicone and dimethicone are added to a suitably sized beaker and heated to a temperature of about 70 degrees C. Stearyl alcohol is added with stirring at 70 degrees C. until it is melted. PEG-8 distearate is added with mixing while maintaining the temperature at 70 degrees C. until it is dissolved. The temperature of the mixture is then increased to about 80 degrees C. Hydrogenated castor oil is added with mixing at 80 degrees C. until it is completely dissolved. The mixture is cooled to about 75 degrees C., the superabsorbent material (Water Lock® Superabsorbent Polymer, A180 from Grain Processing Corporation, Muscatine, Iowa or Hysorb 8100 (BASF, Charlotte, N.C.), and zinc oxide from Elementis Specialties (Hightstown, N.J.). are added with stirring, and the temperature is maintained at 70-75 degrees C. for 15 minutes. The mixture is cooled to about 65 degrees C., farnesol, and fragrance as listed in Table 3 are added with stirring. The mixture is further cooled to about 58 degrees C. and then poured into appropriate containers as described in Example 1.

TABLE 3

| Ingredients(weight %) | Ex. 1 | Ex. 2 | Ex. 3 | Ex. 4 |
|---|---|---|---|---|
| Water Lock ® Superabsorbent, | 20 | 10 | 5 | 20 |
| A180 or Hysorb 8100 | | | | |
| Dimethicone (10 cst) | 10 | 12 | — | 10 |
| C12-15 alkyl benzoate | 5 | — | 7 | 5 |
| Japan Wax Substitute 525 | 3 | — | — | 3 |
| Cyclomethicone 345 | 24.8 | 45.8 | 44.8 | 24.8 |
| Polyethylene (Performalene-400) | 8 | — | 10 | 8 |
| Silicone elastomer (KSG-15) | 25 | — | 15 | 25 |
| Fragrance | 1.2 | 1.2 | 1.2 | 0.6 |
| Stearyl alcohol | — | 20 | — | — |
| Hydrogenated castor oil | — | 4 | — | — |
| PEG-8 distearate | — | 4 | — | — |
| PPG-3 myristyl ether | — | — | 4 | — |
| Farnesol | 1.0 | 1.25 | 0.75 | 1.0 |
| Zinc oxide | 2.0 | 1.75 | 2.25 | 2.0 |
| Polyisobutene 250 | — | — | 5 | — |
| Microencapsulated Fragrance | — | — | — | 0.6 |
| Antiperspirant active (AZZ902) | — | — | 5 | — |
| Total | 100 | 100 | 100 | 100 |

### Example 5

### Soft Solid Product

A soft solid product of about 400 grams may be made using the following ingredients. A silicone elastomer (97% of Dow 9040 from Dow Corning), superabsorbent polymer (2% of the same one used in Example 1), fragrance (1%) along with Farnesol and zinc oxide from Elementis Specialties (Hightstown, N.J.) are combined with mixing in a Hobart mixer at room temperature for about 15-20 minutes.

### Examples 6-9

### Roll-on or Pump Spray Product

A roll-on or pump spray product may be made by combining the following ingredients:

| | Ex. 6 | Ex. 7 | Ex. 8 | Ex. 9 |
|---|---|---|---|---|
| Dimethicone (20 cs) | 46.4 | 46.15 | 46.4 | 46.65 |
| Cyclomethicone 345 | 30 | 30 | 30 | 30 |
| Water Lock ® Superabsorbent, A180 | 15 | 15 | 15 | 15 |
| or Hysorb 8100 | | | | |
| Zinc oxide² | 2 | 2 | 2 | 2 |
| Farnesol | 1.0 | — | — | 0.375 |
| Guaiol | — | 1.25 | — | — |
| Sandalwood oil | — | — | 0.5 | — |
| Cedrol | — | — | — | 0.375 |
| BENTONE ™ 38 Clay¹ | 3.5 | 3.5 | 3.5 | 3.5 |
| Propylene Carbonate | 1.6 | 1.6 | 1.6 | 1.6 |
| Fragrance | 0.5 | 0.5 | 0.5 | 0.5 |
| Encapsulated Fragrance | — | — | 0.5 | — |

### Examples 10-13

### Aerosol Products

Aerosol products may be made by using the following amounts and types of ingredients.

| | Ex. 10 | Ex. 11 | Ex. 12 | Ex. 13 |
|---|---|---|---|---|
| Water Lock ® | 5.0 | 5.0 | 5.0 | 5.0 |
| Superabsorbent, A180 | | | | |
| or Hysorb 8100 | | | | |
| Zinc oxide² | 1.0 | 1.0 | 1.0 | 1.0 |
| Bisabalol | — | — | — | 0.75 |
| Farnesol | 1.0 | 0.9 | — | — |
| Patchouli alcohol | — | 0.1 | — | — |
| Guiac wood oil | — | — | 0.5 | — |
| Cedarwood oil | — | — | 0.5 | — |
| Isopropyl Palmitate | 2.7 | 2.7 | 2.7 | 2.7 |
| BENTONE ™ 38 Clay¹ | 0.25 | 0.25 | 0.25 | 0.25 |
| Propylene Carbonate | 0.08 | 0.08 | 0.08 | 0.08 |
| Fragrance | 0.75 | 0.75 | 0.75 | 0.5 |
| Encapsulated Fragrance | — | — | — | 0.5 |
| Dibutyl Phthalate | 1.2 | 1.2 | 1.2 | — |
| Hydrocarbon Propellant | 86.27 | 86.27 | 86.27 | 86.27 |
| Mixture #2 | | | | |
| MAP-1 Dimethiconol | 1.75 | 1.75 | 1.75 | 1.75 |
| solution in Cyclomethicone | | | | |

¹A quaternium-18 treated hectorite clay available from NL Chemicals Division of NL Industries, Inc. (Hightstown, NJ, USA)
²Any of the zinc oxides described herein may be used.

1. A suspension product for reducing wetness under the arm which product comprises in % by weight based on the entire weight of the product:
(a) 0.01-20 weight % of a superabsorbent polymer selected from the group consisting of a polyacrylate homopolymer (sodium salt) and starch graft copolymers of poly(2-propenamide-co-2-propenioic acid) (sodium salt), with a water absorbing capacity of about 10-1000 g water/g superabsorber as measured in the absence of added salt;
(b) 0.05-2.0 weight % of a sesquiterpene material selected from the group consisting of: (i) a sesquiterpene alcohol; (ii) an essential oil containing at least 5% of a sesquiterpene alcohol; or (iii) a combination of (i) and (ii), provided that the level of sesquiterpene alcohol in the final product is at least 0.05% of the total formula;
(c) 0.05-10 weight % of a small particle size zinc oxide having a particle size range of 0.02-200 microns;
(d) 2-88 weight % of a volatile silicone having a flash point of 100 degrees C. or less;
(e) 0-5 weight % of a surfactant with a hydrophilic/lipophilic balance in the range of 3-13;
(f) 0-5 weight % of an antiperspirant active;
(g) 0-20 weight % of a nonvolatile silicone having a flash point greater than 100 degrees C.; and
(h) 0-20 weight % of an emollient;
(i) 0-5 weight % of a fragrance; and
(j) 0-5 weight % of an encapsulated fragrance; provided that:
(A) if the product is stick or soft solid, the product further comprises a gelling agent selected from the group consisting of 5-30 weight % stearyl alcohol; 0.1-10 weight % (on an actives basis) silicone elastomer; 0.1-20 weight % waxes; 1-3 weight % siliconized polyamides; 1-20 weight % low molecular weight polyethylene having a molecular weight in the range of 400-1000; and combinations of the foregoing;
(B) if the product is an aerosol, the product further comprises 30-90 weight % of a propellant selected from the group consisting of (i) a hydrocarbon, (ii) dimethyl ether, (iii) a hydrofluorocarbon and (iv) mixtures of one or more of (i)-(iii);
(C) if the product is a roll-on or pump spray, the product further comprises 1-15% of a suspending agent selected from the group consisting of:
(i) colloidal silica with a particle size in the range of 2-100 microns;
(ii) clays selected from the group consisting of montmorillonite clays and hydrophobically treated montmorillonite clays, where the clays have a particle size in the range of 50-10,000 nanometers;
(iii) magnesium aluminum silicates with a particle size in the range of 0.1-50 microns; and
(iv) mixtures of any of (i)-(iii).

2. A product as claimed in claim 1 comprising 0. 1-10% of the superabsorbent polymer.

3. A product as claimed in claim 1 comprising 0.1-1.5% of a sesquiterpene containing material.

4. A product as claimed in claim 1 or 3 wherein the sesquiterpene material is a sesquiterpene alcohol selected from the group consisting of:
(i) a cyclic sesquiterpene alcohol selected from the group consisting of:
8-α-acetoxyelemol,
β-acoradienol,
α-acorenol,
β-acorenol,
cis-artenuic alcohol,
β-biotol,
α-bisabolol,
β-bisabolol,
bicyclovitivenol,
β-bisabolenol,
bulensol,
α-cadinol,
epi-α-cadinol,
carotol,
caryophyllene alcohol,
14-hydroxy-9-epi-e-caryophyllene,
5-neo-cedranol,
8-cedren-13-ol,
cedr-8(15)en-9-α-ol,
cedrol,
epi-cedrol,
β-copaen-4-α-ol,
cryptomeridiol,
cubebol,
cubenol,
1-epi-cubenol,
1,10-di-epi-cubenol,
cycloisolongifol-5-ol, dihydrocadinol (1,6-dimethyl-4-iso-propyl-decahydronapthylene)drimenol,
elemol,
epi-globulol,
α-eudesmol,
β-eudesmol,
γ-eudesmol,
7-epi-α-eudesmol,
10-epi-γ-eudesmol,
dihydro-eudesmol,
germacrene D-4-ol,
gleenol,
guaiol,
globulol,
gossonorol,
hexahydrofarnesol (2,6,10-trimethyldodecanol), himachalol,
hinesol,
14-hydroxy-α-humulene,
14-hydroxy-α-muurolene,
5-isocedranol,
isolongifolan-7-α-ol, isolongifolol, khusinol,
ledol,
z-lancetol,
longiborneo,
longifolol,
longipinanol,
cis-muurol-5-en-4-β-ol, cis-muurol-5-en-4-α-ol, α-muurolol,
epi-α-muurenol,
occidentalol,
trans-dihydro-occidentalol, occidol,
e-nuciferol,
patchouli alcohol,
e-β-santalol,
z-α-santalol,
z-β-santalol,
β-santalol,
cis-sesquisabinene hydrate,
trans-sesquisabinene hydrate,
z-sesquilavandulol,
selin-11-en-4-α-ol,
spirosantalol spathulenol,
thujopsan-2-α-ol,
thujopsan-2-β-ol,
turmenol,
valerianol,
viridiflorol,
vitiverol widdrol,
bicyclo-vetiverol, and
tricyclovetiverol; and
(ii) an acyclic sesquiterpene alcohol selected from the group consisting of:
dihydrofarnesol, z,z-farnesol,
e,e-farnesol,
e,z-farnesol,
e-nerolidol,
z-nerolidol,
e-sesquilavandulol,
and tetrahydrofarnesol; and
(iii) a member of the group consisting of sesquiterpene alcohols that have an anti-inflammatory effect as indicated by the ability either in-vitro in a cell culture assay or in-vivo to (A) inhibit prostaglandin cyclooxygenase-I (COX-I), prostaglandin cyclooxygenase-II (COX-II) in-vitro or in-vivo; or (B) the ability to inhibit phospholipase activity, interlukin-1β (II-1β) or interlukin-4 (II-4).

5. A product as claimed in claim 1 wherein the sesquiterpene material is member of the group consisting of: dihydrofarnesol, z,z-farnesol, e,e-farnesol, e,z-farnesol, e-nerolidol, z-nerolidol, e-sesquilavandulol, and tetrahydrofarnesol.

6. A product as claimed in claim 1 wherein the sesquiterpene material is an essential oil.

7. A product as claimed in claim 6 wherein the essential oil is selected from the group consisting of: Patchouli wood oil, Sandalwood oil, Grapefruit oil, Lemongrass oil, Cedarwood oil, and Guiac wood oil.

8. A product as claimed in claim 1 comprising 0.1-5% of the small particle zinc oxide.

9. A product as claimed in claim 1 wherein the volatile silicone is a member of the group consisting of hexamethyl disiloxane, D4 cyclomethicone, D5 cyclomethicone, D6 cyclomethicone, and mixtures of any of the foregoing.

10. A product as claimed in claim 1 comprising 3-13 weight % of the surfactant.

11. A product as claimed in claim 1 further comprising a gelling agent selected from the group consisting of 5-30 weight % stearyl alcohol; 0.1-10 weight % (on an actives basis) silicone elastomer; 0.1-20 weight % waxes; and 0.1-20 weight % low molecular weight polyethylene having a molecular weight in the range of 400-1000.

12. A product as claimed in claim 1 further comprising 1-3% of a gelling agent selected from the group consisting of a siliconized polyamide of Formula IIIA: 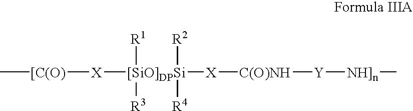
where:
(1) degree of polymerization is a number in the range of 10-40;
(2) n is a number selected from the group consisting of 1-500;
(3) X is a linear or branched chain alkylene having 1-30 carbons;
(4) Y is selected from the group consisting of linear and branched chain alkylenes having 1-40 carbons, wherein:
(A) the alkylene group may optionally and additionally contain in the alkylene portion at least one of the members of a group consisting of (i) 1-3 amide linkages; (ii) C5 or C6 cycloalkane (as a cycloalkylene linkage); and (iii) phenylene optionally substituted by 1-3 members selected independently from the group consisting of C1-C3 alkyls; and
(B) the alkylene group itself may optionally be substituted by at least one member selected from the group consisting of (i) hydroxy; (ii) C3-C8 cycloalkyl; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine; or Y=Z² where 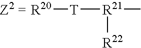
wherein each of R²⁰, R²¹ and R²² are independently selected from the group consisting of linear and branched C1-C 10 alkylenes; and T is selected from the group consisting of (i) a trivalent atom selected from N, P and Al; and (ii) —CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl; and
(5) each of R¹-R⁴ is independently selected from the group consisting of methyl, ethyl, propyl, isopropyl, a siloxane chain, and phenyl, wherein the phenyl may optionally be substituted by 1-3 members from the group consisting of methyl and ethyl;
wherein the polyamide of Formula IIIA has:
(i) a silicone portion in the acid side of the polyamide;
(ii) a degree of polymerization in the range of 10-40;
(iii) an average molecular weight of at least 50,000 daltons with at least 95% of the polyamide having a molecular weight greater than 10,000 daltons; and
(iv) a polydispersity of less than 20.

13. A product as claimed in claim 1 further comprising 0.05-50 weight % of a silicone copolyol which is 10% in cyclomethicone, or its equivalent.

14. A product as claimed in claim 1 further comprising further comprising 1-3 weight % of an aluminum or aluminum/zirconium antiperspirant salt.

15. A product as claimed in claim 1 further comprising particularly 5-10% of a nonvolatile silicone having a flash point greater than 100 degrees C.

16. A product as claimed in claim 1 further comprising 2-12% of an emollient.

17. A product as claimed in claim 16 wherein the emollient is selected from the group consisting of C12-15 alkyl benzoate, PEG-8 distearate, PPG-3-myristyl ether, and polyisobutene 250.

18. A product as claimed in any one of claims 1-17 which is free of one or more of surfactant, antiperspirant active, non-volatile silicone, fragrance and microencapsulated fragrance.

19. A product as claimed in any one of claims 1-17 which is free of antiperspirant actives selected from the group consisting of aluminum and aluminum/zirconium salts.

20. A product as claimed in claim 1 wherein the product is a stick and comprises:
(a) 8-25 weight % of the superabsorbent polymer;
(b) 0.05-10 weight % of the sesquiterpene material;
(c) 0.05-10 weight % of the small particle size zinc oxide;
(d) 10-25 weight % of the gellant;
(e) 40-70 weight % of the volatile silicone;
(f) 0-1 5 weight % of a non-volatile silicone which is a dimethicone having a viscosity in the range of 6-1000 centistokes;
(g) 2-10 weight % of an emollient selected from the group consisting of polyisobutene, and C12-15 alkyl benzoates;
(h) 0-5 weight % fragrance;
(i) 0-5 weight % of a microencapsulated fragrance;
(j) 0-10 weight % of a surfactant; and
(k) less than 2 weight % water.

21. A product as claimed in claim 1 wherein the product is a soft solid and comprises:
(a) 70-99.94 weight % of the silicone elastomer;
(b) 0.01-30 weight % of the superabsorbent;
(c) 0.05-10 weight % of the sesquiterpene material;
(d) 0.05-10 weight % of the small particle size zinc oxide;
(e) 0-5 weight % of a fragrance;
(f) 0-5 weight % of a microencapsulated fragrance; and (g) less than 2 weight % water.

22. A product as claimed in claim 1 wherein the product is a roll-on or pump spray and comprises:
(a) 0-80 weight % of the volatile silicone;
(b) 0-50 weight % of the silicone elastomer;
(c) 0-80 weight % of the non-volatile silicone which is a dimethicone having a viscosity in the range of 6-1000 centistokes;
(d) 0.01-30 weight % of the superabsorbent;
(e) 0.05-10 weight % of the sesquiterpene material;
(f) 0.05-10 weight % of the small particle size zinc oxide;
(g) 0-5 weight % fragrance;
(h) 0-5 weight % of a microencapsulated fragrance;
(i) 1-10% of a suspending agent; and
(j) less than 2 weight % water.

23. A product as claimed in claim 1 wherein the product is an aerosol and comprises:
(a) 30-80 weight % of the volatile silicone;
(b) 30-90% of an aerosol propellant;
(c) 0.01-30 weight % of the superabsorbent;
(d) 0.05-10 weight % of the sesquiterpene material;
(e) 0.05-10 weight % of the small particle size zinc oxide;
(f) 0-5 weight % fragrance;
(g) 0-5 weight % of a microencapsulated fragrance;
(h) 1-10% of a suspending agent; and
(i) less than 2 weight % water.
